(19) United States
(12) Patent Application Publication
(10) Pub. No.: US 2014/0221963 A1
(43) Pub. Date: Aug. 7, 2014

(54) USE OF EATING DETECTION TO CONTROL THE RELEASE OF BIOLOGICALLY ACTIVE AGENTS

(71) Applicants: Edy E. Soffer, Los Angeles, CA (US); Jeffrey Conklin, Los Angeles, CA (US)

(72) Inventors: Edy E. Soffer, Los Angeles, CA (US); Jeffrey Conklin, Los Angeles, CA (US)

(21) Appl. No.: 14/239,764

(22) PCT Filed: Aug. 23, 2012
(86) PCT No.: PCT/US2012/052117

§ 371 Date: Feb. 19, 2014

Related U.S. Application Data
(60) Provisional application No. 61/527,254, filed on Aug. 25, 2011.

(51) Int. Cl. A61M 5/172; A61F 5/00

(52) U.S. Cl. CPC A61M 5/1723; A61F 5/003; A61F 2005/0016; A61M 2202/07; A61M 2205/04; A61M 2230/08
USPC 604/503; 604/66

(57) ABSTRACT

Systems, apparatuses, and methods for delivery of an active agent in response to the changes in electrical activity of a subject's lower esophageal sphincter. Changes in electrical activity can be monitored by a monitoring unit containing a microprocessor. A reservoir and pump can be used to store and release the active agent. The active agent can be cholecystokinin, peptide YY, glucagon-like peptide 1 or 2, and ghrelin, or a combination thereof.

### CROSS-REFERENCE TO RELATED APPLICATIONS

This application claims the benefit of Provisional Patent Application No. 61/527,254, filed on Aug. 25, 2011, which is incorporated by reference herein.

### BACKGROUND

1. Field of Invention

This invention relates to systems and methods utilizing automatic eating detection (AED) to control the release of biologically active agents.

2. Related Art

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Certain conditions, such as obesity, can benefit from treatment that is delivered in conjunction with food ingestion. Currently, obesity is treated by medical and surgical means. Medically, a number of agents are in use, or being investigated for the treatment of obesity. Some of these agents are given for the purpose of reducing the desire to eat, or to induce a feeling of fullness with eating, that results in the consumption of a smaller than usual amount of food. Such agents, taken either by mouth or by injection, may be active for prolonged periods of time, including periods when food is not being consumed, such as during sleep.

### SUMMARY

In one embodiment, a method for controlling the release of a biologically active agent to a tissue of a subject is provided. The method includes releasing the agent to the tissue in response to changes in electrical activity of the subject's lower esophageal sphincter.

In a more particular embodiment, the method further includes monitoring the changes in electrical activity prior to releasing the agent. In addition, the agent can be released from a storage reservoir located in the subject.

In yet another embodiment, the tissue is selected from a group consisting of subcutaneous, adipose, muscle, and vein.

In some embodiments, the changes in the electrical activity indicate that the subject has started consuming food or drink, is consuming food or drink, has stopped consuming food or drink, has consumed food or drink, or any combination thereof.

In a more particular embodiment, the changes in electrical activity comprise changes in electrical amplitude or duration. In another embodiment, an increase in amplitude to a value greater than baseline indicates food or drink intake. In yet another embodiment an about three to about four fold increase in amplitude from baseline indicates food or drink intake.

In the method, an amplitude of about 0.30 mV to about 0.90 mV, or about two-fold increase in amplitude from baseline, indicates a dry swallow. In yet another embodiment, an amplitude of about 0.31 mV to about 1.03 mV, or an about two-fold increase in amplitude from baseline, indicates a wet swallow. In one embodiment, an amplitude of about 0.55 mV to about 1.57 mV, or a greater than three-fold increase in amplitude from baseline, or an about three to about four fold increase in amplitude from baseline, indicates solid food intake.

In some embodiments, the above method is used on a subject undergoing treatment for obesity, treatment to prevent obesity, treatment for diabetes, and/or treatment for an eating disorder.

In the method, the monitoring can comprise monitoring the electrical activity by use of a microprocessor, or monitoring the electrical activity by use of one or more electrodes positioned within, in contact with or proximate to the gastroesophageal junction of the subject, or a combination thereof. It is understood that “positioned within, in contact with or proximate to” includes any combination of such positioning.

In another aspect, a system usable with the foregoing methods, for controlling the release of a biologically active agent in a subject, is provided. The system includes a monitoring unit that monitors electrical activity changes of the subject's lower esophageal sphincter; and a reservoir that releases an active agent to the subject in response to a signal from the monitoring unit about the electrical activity changes. The reservoir can be implantable on or within the subject, or can be for external use, or a combination thereof. The system can further include one or more pumps.

In another embodiment, the monitoring unit includes a microprocessor that monitors electrical activity and detects eating and/or generate electrical signals based on the monitored electrical activity. In yet another embodiment, the system further includes a recording module for recording electrical data based on the monitored electrical activity; and/or a pulse generator and a recording module.

In a further aspect, the system includes a computer for receiving electrical signals, analyzing electrical signals, processing electrical signals, and sending a signal regarding the electrical signals to another system, computer or device. The system can further comprise one or more electrodes for detecting the electrical activity changes and positionable within, in contact with or proximate to the gastroesophageal junction of the subject.

The system can include any combination of the monitoring unit, reservoir, recording module, pulse generator, computer, one or more electrodes, one or more pumps, and circular wheel and watch-type drive mechanism.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts a schematic representation of various components of a system for controlled release of agents in conjunction with eating detection.

FIG. 2 depicts a schematic representation of a microprocessor and agent delivery system in conjunction with eating detection.

FIG. 3 depicts a representative location for detection of LES electrical activity for use in accordance with a system for controlled release of agents in conjunction with eating detection.

### DETAILED DESCRIPTION

The inventors recognize that a need exists for systems and methods that deliver agents in conjunction with food consumption. Delivery of biologically active agents in conjunction with meals can reduce adverse events and improve the efficacy of therapy. Biologically active agents are compounds that can affect biological function or behavior. Examples of biologically active agents include, but are not limited to, cholecystokinin, peptide YY, glucagon-like peptides 1 and 2, and ghrelin. A biologically active agent can be a hormone, peptide, polypeptide, small-molecule compound, drug or pharmaceutical agent, or a combination thereof, which is biologically active.

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention.

In one aspect, apparatuses, systems and methods that use AED to control delivery of one or more agents in conjunction with food consumption are provided. This can involve the detection of electrical activity changes in the lower esophageal sphincter (LES) of a subject that are indicative of eating, and the automatic delivery of an active agent to the subject based on, or in response to, the electrical activity changes. Examples of subjects that can benefit from these apparatuses, systems and methods include but are not limited to obese subjects treated with agents that control food intake, or diabetic patients treated with insulin. The subject can be a human or an animal.

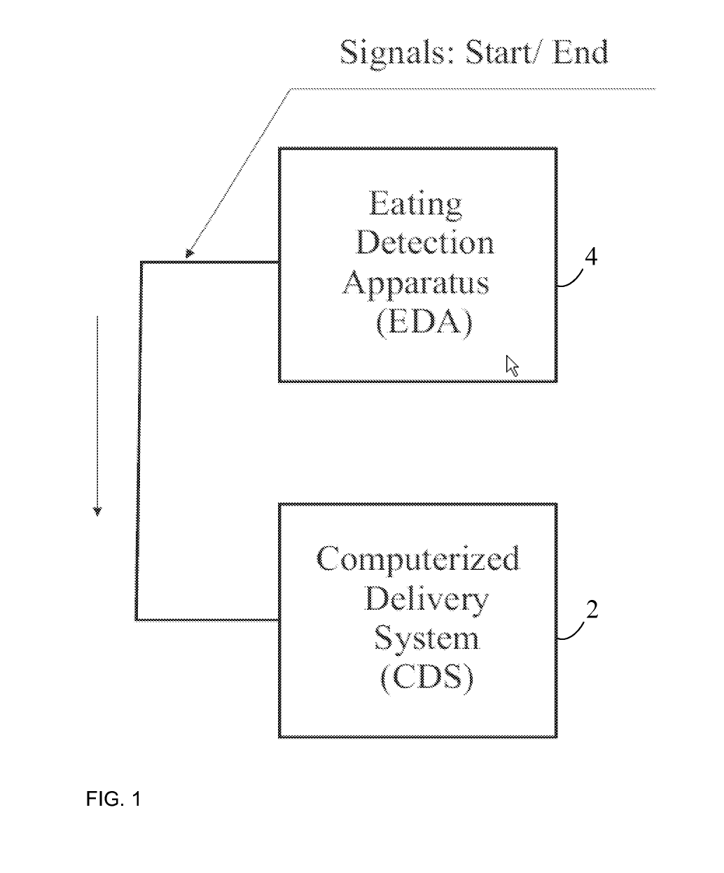

Referring to FIG. 1, embodiments of the present invention provide for a system, comprising a computerized delivery system 2 and an eating detection sub-system 4.

The system can be placed by laparoscopy.

### Computerized Delivery System

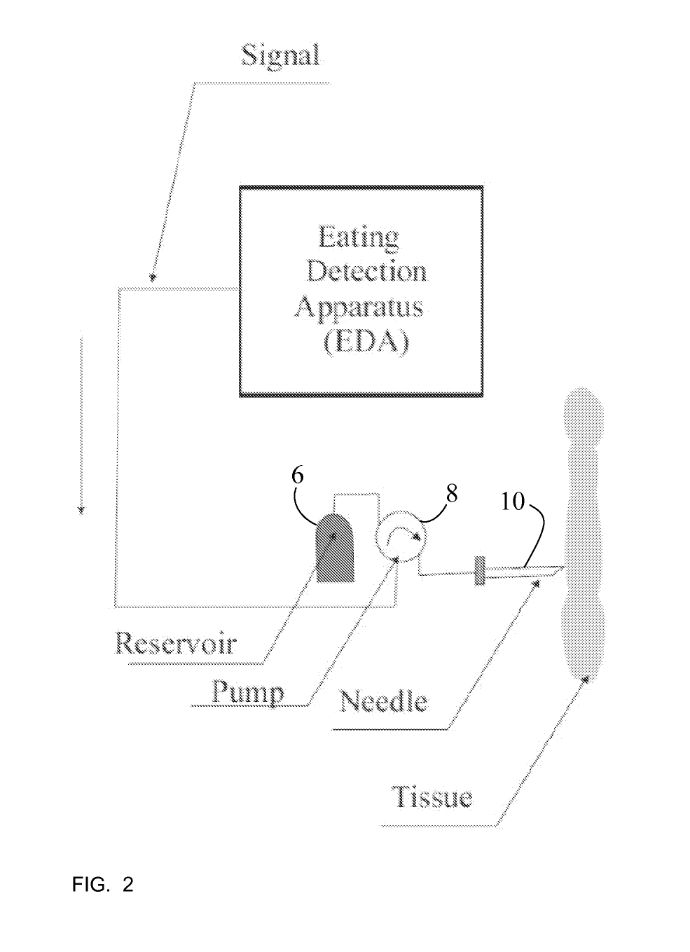

In various embodiments, a computerized delivery system (CDS) can use a microprocessor to control the system. In various embodiments, the CDS will cause a delivery of an agent to a tissue, in accordance with a subject's eating process detected by an AED-system. Implantable drug delivery devices are currently used to administer therapeutic agents to various locations within the body. A number of reservoirs, pumps and combinations of reservoirs and pumps have been developed. Referring to FIG. 2, a typical device includes a reservoir 6 to store the agent and a separate pump 8 or other flow control device to deliver the agent. The system is arranged so that the pump pulls the agent from the reservoir via a passageway between the reservoir and pump. After passing through the pump, the agent is delivered via an outlet line, such as a needle 10 or catheter, to a desired location within the patient's body. Examples of pumps include peristaltic, piston or piezoelectric micropump-based devices.

In other systems, implantable electromechanical drug delivery devices can include, within a fluid impermeable and sealed casing, a watch-type drive mechanism that drives a circular wheel. The wheel contains a plurality of cavities, all of which apparently are radially disposed in a single diametral plane about the circumference of the wheel. Once the drug-containing cavity moves into alignment with an aperture through the casing, a piston associated with the cavity ejects medicine out of the cavity and through the aperture. Implantable drug delivery devices can include a reservoir, a dispensing chamber adjacent to the reservoir, a dispensing passage provided along an interior surface of the dispensing chamber, and an actuator for applying a moving compressive force onto the dispensing passage. As a compressive force applied by the actuator moves along the dispensing passage, drug is simultaneously ejected out of the dispensing passage into a catheter for delivery to a target site, and additional drug is drawn into the dispensing passage from the reservoir. Microchip based drug delivery devices can include a plurality of drug reservoirs etched into a substrate, for example, a single microchip. Drugs then are sealed within each of the reservoirs with a seal. The seal can be a material that dissolves upon application of an electric potential. An article by Santini et al, (1999) Nature 397: 335-338, (incorporated by reference herein) describes a solid-state silicon microchip that provides controlled release of a drug of interest via electrochemical dissolution of a thin membrane covering a micro-reservoir filled with drug.

The drug delivery device can be a drug delivery device described in any of the following patents: U.S. Pat. No. 3,692,027, U.S. Pat. No. 6,283,949 B1, U.S. Pat. No. 5,797,898, U.S. Pat. No. 6,928,338, U.S. Pat. No. 6,582,418, U.S. Pat. No. 6,190,359, and U.S. Pat. No 6,123,861, all of which are incorporated by reference herein.

The CDS can comprise a small storage reservoir and a pump (see FIG. 2). The microprocessor can detect electrical recording, process algorithms and signals to activate the pump, which then releases an agent to a tissue. In various embodiments the tissue could be subcutaneous, in others it could be adipose tissue, muscle, or a vein. In one embodiment, the CDS can be placed in the subcutaneous tissue, in others it can be placed in adipose tissue. The CDS could be place on the skin, in subcutaneous tissue, in adipose tissue or other body spaces.

A built-in algorithm controls the computerized hydraulic system to function at the desired stage. The algorithm can be individualized, so as to provide the appropriate detection for each subject. (See Sanmiguel et al. The Effect of Eating on Lower Esophageal Sphincter Electrical Activity. Am J. Physiology 2009; 296: G793-G797, incorporated by reference herein.) The first step is eating detection. A change in the amplitude of the electrical activity in the LES region is detected by the implanted electrodes connected to a microprocessor with a dedicated algorithm. The algorithm is based on the fact that the amplitude of electrical activity increases when food is consumed, and is highest with solid food compared to liquids, or swallows of saliva. This detection mechanism can be individualized and adjusted to the response of each subject. When eating is detected, a signal can be sent by the microprocessor to activate the pump, resulting in delivery of a predetermined amount of agent to the tissue. In various embodiments, the signal can be sent by the microprocessor immediately after detecting food intake.

### Automatic Detection of Eating

The eating detection sub-system can act as a controller of the system. After the detection of eating, a signal is sent to the pump to cause delivery of an agent.

Various embodiments of the present invention utilize systems and methods of automatic detection of eating to initiate the control of the gastric band. In various embodiments, the automatic detection of eating can utilize the methods, devices, and systems set for the in U.S. Patent Application Publication no. 2010/0076345, which is hereby incorporated by reference as though fully set forth in its entirety.

Electrical activity of the lower esophageal sphincter has been recorded and studied. Swallowing produces changes in the motor activity of the LES. The inventors believe that these changes are related to specific changes in LES electrical activity. The beginning and duration of a meal can be identified by distinct, easily recognizable changes in the amplitude of LES electrical activity. These changes also depend on the type of substance being swallowed (e.g., saliva, liquid and solids), and are most prominent with solid food. Further, during fasting, transient increases in LES electrical activity not related to swallowing do not produce the same increase in electrical activity as seen during swallowing of food. Thus, changes in LES electrical activity can be used for eating detection.

The method of detecting food or drink intake in a subject can comprise: a) placing one or more electrodes in contact with or proximate to the subject's LES; and b) identifying food or drink intake by monitoring electrical activity in or proximate to the LES using one or more electrodes. In various embodiments, bipolar electrodes can be used and thus, only one electrode is necessary.

The lower esophageal sphincter is a ring of muscle tissue located at the bottom of the esophagus where the esophagus meets the stomach. Normally, the LES acts as a valve to prevent the backflow of stomach contents into the esophagus. The junction between the esophagus and the stomach is called the gastroesophageal junction.

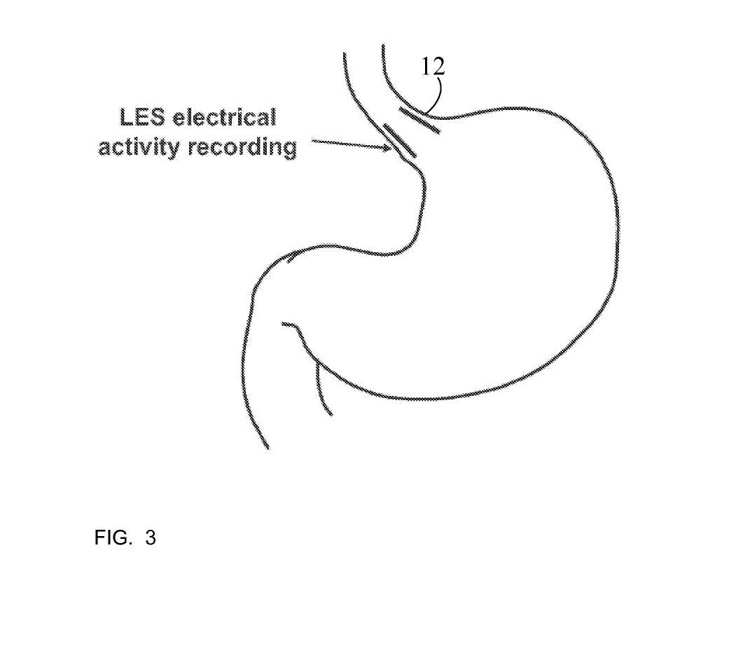

Referring to FIG. 3, in one embodiment, one or more electrodes are placed in contact with the LES 12 or in contact with a proximate region to the LES and the electrical activity is monitored at that location (see FIG. 3). An increase in the amplitude of electrical activity in the monitored location indicates food or drink intake, and a decrease in the amplitude back to about baseline level indicates the cessation of food or drink intake. Further, the degree of the change in amplitude (e.g., increase in amplitude) can be used to differentiate between types of swallows (saliva, liquid or solid food). The duration of change in amplitude helps to determine the length of period of food consumption. For example, a short duration indicates simple swallows or a very small snack and a long duration indicates the consumption of a larger meal. The electrical activity of the LES while in a resting or non-swallowing state can establish the baseline level, and amplitudes above the baseline can indicate dry swallows, wet swallows, or solid food swallows, depending on the size and duration of the amplitudes.

In some embodiments, a pair of electrodes is placed. Two electrodes can be positioned at opposite sides of the gastroesophageal junction (GEJ). In particular embodiments, one electrode is positioned in the anterior aspect of the GEJ and a second electrode is positioned in the posterior aspect of the GEJ.

In other embodiments, one or more electrodes are positioned away from the vagus nerve trunks. In a particular embodiment, one or more electrodes are positioned as far away from the vagus nerve trunks as possible so long as electrical activity indicative of food or drink intake can be detected. In a particular embodiment, two electrodes are positioned as far away from the vagus nerve trunks as possible.

An electrode can be of any size suitable for placement on or in the LES, or on or in a proximate region to the LES. In various embodiments, the electrodes can be about 1 mm long to about 50 mm long, about 5 mm long to about 25 mm long, or about 10 mm long to about 20 mm long. In one embodiment, the electrode can be about 15 mm long. The electrode can also be of any shape suitable for placement on the LES or on a proximate region to the LES; for example, circular, square, rectangular, etc. The electrode can also be of any dimension suitable for placement on the LES or on a proximate region to the LES. The electrode can be attached on the surface of the LES or proximate region, or implanted into the LES or proximate region.

Placing an electrode in contact with the LES or proximate to the LES, or both, can be performed by any method known in the art; for example, by a surgical procedure or by an endoscopic procedure. The electrode can be placed on any level in the LES tissue from the inner lining (i.e., mucosa) to the muscle layer. In one particular embodiment, an electrode can be sutured to a muscle layer of the LES or a proximate region to the LES.

In some embodiments, monitoring the electrical activity comprises detecting the electrical activity in the LES. In particular embodiments, monitoring the electrical activity comprises measuring the amplitude and/or duration of the electrical activity in the LES.

An increase in amplitude of the monitored electrical activity to a value greater than baseline amplitude can indicate food or drink intake. In certain embodiments, an about three to about four fold increase in amplitude from baseline amplitude indicates food or drink intake.

In some embodiments, an amplitude of about 0.30 mV to about 0.90 mV indicates a dry swallow, or an amplitude of about 0.40 mV to about 0.80 mV, about 0.45 mV to about 0.75 mV, or about 0.5 mV to about 0.7 mV indicates a dry swallow. In a particular embodiment, an amplitude of about 0.6 mV indicates a dry swallow. Alternatively, an about two-fold increase in amplitude indicates a dry swallow. A “dry swallow” is a swallow in the absence of food or drink.

In some embodiments, an amplitude of about 0.31 mV to about 1.03 mV indicates a drink intake (wet swallow), or an amplitude of about 0.43 mV to about 0.91 mV, about 0.52 mV to about 0.88 mV, or about 0.58 mV to about 0.82 mV indicates a drink intake. In a certain embodiment, an amplitude of about 0.7 mV indicates a drink intake. Alternatively, an about two-fold increase in amplitude indicates a wet swallow.

In some embodiments, an amplitude of about 0.55 mV to about 1.57 mV indicates solid food intake, or an amplitude of about 0.72 mV to about 1.4 mV, about 0.81 mV to about 1.32 mV, or about 0.89 mV to about 1.23 mV indicates solid food intake. In a particular embodiment, an about 1.06 mV indicates solid food intake. Alternatively, a greater than three-fold increase in amplitude indicates solid food intake, or an about three to about four fold increase in amplitude indicates solid food intake.

The specific amplitudes indicative of dry swallows, wet swallows and food intake will vary depending on the subject being examined. The range of amplitudes for a specific subject can be obtained by measuring the subject's background level of electrical activity while the subject is in a resting or non-swallowing state, then measuring the amplitudes when the subject is performing a dry swallow, is swallowing liquid, and is swallowing solid food. These observed amplitudes can be used to identify background electrical activity and different types of swallows when the subject is subsequently monitored for food or drink intake.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, an about three to about four fold decrease in amplitude from the increased amplitude indicates food or drink intake has stopped. In certain embodiments, an amplitude of about 0.135 mV to about 0.495 mV indicates that food or drink intake has stopped, or an amplitude of about 0.195 mV to about 0.435 mV, about 0.225 mV to about 0.405 mV, or about 0.255 mV to about 0.375 mV indicates that food or drink intake has stopped. In a particular embodiment, an amplitude of about 0.315 mV indicates that food or drink intake has stopped. In certain embodiments, detection of cessation of eating will result in a signal to stop delivery of an agent.

Data on electrical activity in the LES can be transmitted to a recording/analyzing device, such as a microprocessor incorporated in the CDS, by way of electrodes. In another embodiment, a miniaturized recorder implanted in the LES or in contact with a proximate region to the LES can transmit data in a wireless fashion to an implanted system, or to an outside device.

In some embodiments, a signal indicating that a subject has started consuming food or drink, is in the process of consuming food or drink, has stopped consuming food or drink, has consumed food or drink, or any combination thereof, can be generated based on the amplitude and duration of the electrical activity of the LES or proximate to the LES. The signal can be sent to a receiving device, such as a computer or a system containing a receiving device, or other device or system associated with food or drink intake or the cessation of food or drink intake. As such, additional embodiments can further comprise using a receiver to receive signals regarding the subject's food or drink intake. In some embodiments, the receiving device is used in a clinical application associated with food or drink intake. Thus, the detection of food or drink intake or cessation of food or drink intake, or signals indicative thereof, may be used in conjunction with other technology for clinical applications. That is, the detection of food or drink intake or the cessation of food or drink intake, or signals indicative thereof, can be used to trigger an intervention treatment that is associated with the food or drink intake or the cessation of food or drink intake.

Additional embodiments can further comprise using a computer or computer system to perform a number of functions, for example, including but not limited to receiving electrical signals, analyzing electrical signals, processing electrical signals, and sending a signal regarding the received, analyzed and/or processed electrical signals to another system, computer or device. Such computers and computer systems are known in the art and one of skill in the art will be able to determine, without undue experimentation, a computer or a computer system that is suitable for such use.

A device for practicing the method of detecting food or drink intake can comprise: a) one or more electrodes, for monitoring electrical activity of the subject's LES or a region proximate to the LES; and a microprocessor that can monitor electrical activity and incorporates an algorithm that detect eating and generate electrical signals based on the monitored electrical activity. Such microprocessor can be part of a pulse generator. The device can further comprise a recording module, for recording electrical data based on the monitored electrical activity. The one or more electrodes can be functionally connected to the microprocessor/pulse generator or the recording module, or both, and the device can be configured to identify food or drink intake by monitoring electrical activity of or proximate to the LES using the one or more electrodes. In some embodiments, one or more pairs of electrodes is utilized. The detection device can be configured to automatically detect food or drink intake in a subject.

In one embodiment, the detection device comprises one or more electrodes, and a microprocessor, wherein the one or more electrodes are connected to the microprocessor that can analyze electrical signals from the LES or a region proximate to the LES and also send electrical signals to activate the hydraulic system. The one or more electrodes can be one or more pairs of electrodes, or be a single lead (e.g., bipolar electrode). In particular embodiments, the detection device is configured to measure the amplitude and/or duration of the electrical activity in the LES or in the proximate region to the LES. In some embodiments, the detection device is an implantable device.

In one embodiment the whole system is implantable. In another embodiment, the microprocessor is positioned outside the body, and both recording of electrical signals from the LES or a region proximate to the LES and delivery of signals to the hydraulic system can be done by wireless connections. In some embodiments, a recording of the electrical activity is obtained by placing wands on the subject's skin that detect the electrical activity, and connecting the wands to data loggers.

The electrode can be any size suitable for placement on the LES or a proximate region to the LES. In various embodiments, the electrodes can be about 1 mm long to about 50 mm long, about 5 mm long to about 25 mm long, or about 10 mm long to about 20 mm long. In one embodiment, the electrode may be about 15 mm long. The electrode can be any shape suitable for placement at the LES; for example, circular, square, rectangular, etc., and can be any dimension suitable for placement at the LES.

In some embodiments, the detection device can further comprise a computer. The computer can be used to perform a number of functions; for example, including but not limited to receiving electrical signals, analyzing electrical signals, processing electrical signals, and sending a signal regarding the received, analyzed and/or processed electrical signals to another system, computer or device.

In one embodiment, the detection device is configured to generate and send a signal to another device indicating the electrical activity of the LES. In some embodiments, the signal can be a signal that indicates that the subject has started consuming food or drink, is consuming food or drink, has stopped consuming food or drink, has consumed food or drink, or any combination thereof.

In some embodiments, the detection device is configured to generate and send a signal when an increase in amplitude from baseline amplitude is detected. In another embodiment, the device is configured to generate and send a signal that the subject has consumed food or drink when an about three to about four fold increase in amplitude from a baseline amplitude is detected.

In some embodiments, the detection device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the detection device is configured to generate and send a signal that the subject has consumed a liquid when an amplitude of about 0.31 mV to about 1.03 mV, about 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when an amplitude of about 0.7 mV has been detected. Alternatively, the device is configured to generate and send a signal that the subject has consumed a liquid when an about two-fold increase in amplitude has been detected.

In some embodiments, the detection device is configured to generate and send a signal that the subject has consumed solid food when an amplitude of 0.55 mV to about 1.57 mV has been detected. In certain embodiments, the device is configured to generate and send a signal that the subject has consumed food when amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the detection device is configured to generate and send a signal that the subject has ceased consuming food or drink when a reversion of the increased amplitude back to approximately baseline amplitude is detected. In certain embodiments, the device is configured to generate and send a signal that the subject has stopped consuming food or drink when an about three to about four fold decrease in amplitude from the increased amplitude is detected.

In some embodiments, the detection device can be configured to generate and send a signal that the subject has ceased consuming food or drink when an amplitude of about 0.135 mV to about 0.495 mV has been detected. In particular embodiments, the device may be configured to generate and send a signal that the subject has ceased consuming food or drink when amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected.

These signals may be useful for a variety of clinical applications. The signals may be used in conjunction with other technology for clinical applications. That is, the signal generated when food or drink intake is detected or when the cessation of food or drink intake is detected may be used to trigger an intervention treatment that is associated with the food or drink intake or the cessation of food or drink intake.

A system for practicing the method can comprise the following. A pair of electrodes is implanted in the lower esophageal sphincter (LES) at the level of the gastro-esophageal junction. The electrodes are connected to a microprocessor. The microprocessor receives and processes signals from the electrodes regarding the subject's intake of food and drink. The microprocessor can send a signal to a pump. All of these components can be manufactured separately, in combination, or as a single device.

In one embodiment, the system comprises a device for monitoring the electrical activity of the LES and a computer for interpreting and/or recording the electrical activity of the LES. In another embodiment, the system further comprises a device for recording the electrical activity of the LES. The device for monitoring the electrical activity can comprise one or more electrodes, a pulse generator, and a recording module, wherein the pulse generator or the recording module, or both, can be connected to the one or more electrodes and the device is configured to measure the electrical activity in the LES or in a proximate region to the LES. In particular embodiments, the device for monitoring the electrical activity is configured to measure the amplitude and/or duration of the electrical activity in the LES or in the proximate region to the LES.

The electrode can be any size suitable for placement at the LES. In various embodiments, the electrode can be about 1 mm long to about 50 mm long, about 5 mm long to about 25 mm long, or about 10 mm long to about 20 mm long. In one embodiment, the electrode is about 15 mm long. The electrode can be any shape suitable for placement at the LES, such as circular, square, rectangular, etc. The electrode can also be of any dimension suitable for placement at the LES.

A computer can be used to perform a number of functions, for example, including but not limited to receiving electrical signals, analyzing electrical signals, processing electrical signals, and sending a signal regarding the electrical signals to another system, computer or device

Additional embodiments of the system further comprise a receiver for receiving signals regarding a subject's food or drink intake.

The system can comprise a device for monitoring the electrical activity and a device for sending a signal to a second system or device. In one embodiment, the second system or device is a system or device for the treatment of obesity.

The device for sending a signal to a second system or device can be configured to generate and send a signal to indicate the electrical activity of the LES. In particular embodiments, the signal is a signal indicating that the subject has started consuming food or drink, is in the process of consuming food or drink, has stopped consuming food or drink, has consumed food or drink, or any combination thereof.

In some embodiments, the device for sending a signal is configured to generate and send a signal when an increase in amplitude from baseline amplitude is detected. In certain embodiments, the device is configured to generate and send a signal that the subject has consumed food or drink when an about three to about four fold increase in amplitude from a baseline amplitude is detected.

In some embodiments, the device is configured to generate and send a signal that the subject has swallowed when an amplitude of about 0.30 mV to about 0.90 mV has been detected. In particular embodiments, the device is configured to generate and send a signal that the subject has swallowed when an amplitude of 0.40 mV to about 0.80 mV, about 0.45 mV to about 0.75 mV, or about 0.5 mV to about 0.7 mV has been detected. In a particular embodiment, the device is configured to generate and send a signal that the subject has swallowed when an amplitude of about 0.6 mV has been detected. Alternatively, the device is configured to generate and send a signal that the subject has swallowed when an about two-fold increase in amplitude has been detected.

In some embodiments, the device is configured to generate and send a signal that the subject has consumed a liquid when amplitude of about 0.31 mV to about 1.03 mV has been detected. In other embodiments, the device is configured to generate and send a signal that the subject has consumed a liquid when amplitude of 0.43 mV to about 0.91 mV, about 0.52 mV to about 0.88 mV, or about 0.58 mV to about 0.82 mV has been detected. In a particular embodiment, the device is configured to generate and send a signal that the subject has consumed a liquid when amplitude of about 0.7 mV has been detected. Alternatively, the device is configured to generate and send a signal that the subject has consumed a liquid when an about two-fold increase in amplitude has been detected.

In some embodiment, the device is configured to generate and send a signal that the subject has consumed food when an amplitude of 0.55 mV to about 1.57 mV has been detected. In other embodiments, the device is configured to generate and send a signal that the subject has consumed food when an amplitude of about 0.72 mV to about 1.4 mV, about 0.81 mV to about 1.32 mV, or about 0.89 mV to about 1.23 mV has been detected. In a particular embodiment, the device is configured to generate and send a signal that the subject has consumed food when an amplitude of about 1.06 mV has been detected. Alternatively, the device is configured to generate and send a signal that the subject has consumed food when greater than a two-fold increase in amplitude has been detected. In particular embodiments, the device is configured to generate and send a signal that the subject has consumed food when an about three to about four fold increase in amplitude is detected.

Reversion of an increased amplitude back to baseline or to a value of approximately baseline amplitude can indicate that food or drink intake has stopped. Further, a decrease in amplitude from a higher value to a lower value can indicate that food or drink intake has stopped. In some embodiments, the device is configured to generate and send a signal that the subject has ceased consuming food or drink when a reversion of the increased amplitude back to approximately baseline amplitude is detected. In particular embodiments, the device is configured to generate and send a signal that the subject has stopped consuming food or drink when an about three to about four fold decrease in amplitude from the increased amplitude is detected.

In some embodiment, the device is configured to generate and send a signal that the subject has ceased consuming food or drink when an amplitude of about 0.135 mV to about 0.495 mV has been detected. In certain embodiments, the device is configured to generate and send a signal that the subject has ceased consuming food or drink when an amplitude of about 0.195 mV to about 0.435 mV, about 0.225 mV to about 0.405 mV, about 0.255 mV to about 0.375 mV has been detected. In a particular embodiment, the device is configured to generate and send a signal that the subject has ceased consuming food when an amplitude of about 0.315 mV has been detected

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well.

What is claimed is:

1. A method for controlling the release of a biologically active agent to a tissue of a subject, comprising
releasing said agent to the tissue in response to changes in electrical activity of the subject's lower esophageal sphincter.

2. The method of claim 1, further comprising monitoring the changes in electrical activity prior to releasing the agent.

3. The method of claim 1, wherein said agent is released from a storage reservoir located in the subject.

4. The method of claim 1, wherein the tissue is selected from a group consisting of subcutaneous, adipose, muscle, and vein.

5. The method of claim 1, wherein the changes in the electrical activity indicate that the subject has started consuming food or drink, is consuming food or drink, has stopped consuming food or drink, has consumed food or drink, or any combination thereof.

6. The method of claim 1, wherein said changes in electrical activity comprising changes in electrical amplitude or duration.

7. The method of claim 6, wherein an increase in amplitude to a value greater than baseline indicates food or drink intake.

8. The method of claim 6, wherein an about three to about four fold increase in amplitude from baseline indicates food or drink intake.

9. The method of claim 6, wherein an amplitude of about 0.30 mV to about 0.90 mV, or about two-fold increase in amplitude from baseline, indicates a dry swallow.

10. The method of claim 6, wherein an amplitude of about 0.31 mV to about 1.03 mV, or an about two-fold increase in amplitude from baseline, indicates a wet swallow.

11. The method of claim 6, wherein an amplitude of about 0.55 mV to about 1.57 mV, or a greater than three-fold increase in amplitude from baseline, or an about three to about four fold increase in amplitude from baseline, indicates solid food intake.

12. The method of claim 6, wherein an amplitude of about 0.55 mV to about 1.57 mV, or a greater than three-fold increase in amplitude from baseline, or an about three to about four fold increase in amplitude from baseline, indicates solid food intake.

13. The method of claim 1, wherein the subject is undergoing treatment for obesity, treatment to prevent obesity, treatment for diabetes, and/or treatment for an eating disorder.

14. The method of claim 1, wherein the biologically active agent comprises a hormone, peptide, polypeptide, small-molecule compound, drug or pharmaceutical agent, or a combination thereof.

15. The method of claim 1, wherein the biologically active agent is selected from a group consisting of cholecystokinin, peptide YY, glucagon-like peptide 1 or 2, and ghrelin, or a combination thereof.

16. The method of claim 1, wherein the monitoring comprises monitoring the electrical activity by use of a microprocessor, or monitoring the electrical activity by use of one or more electrodes positioned within, in contact with or proximate to the gastroesophageal junction of the subject, or a combination thereof.

17. A system for controlling the release of a biologically active agent in a subject, comprising a monitoring unit that monitors electrical activity changes of the subject's lower esophageal sphincter; and a reservoir that releases an active agent to the subject in response to a signal from the monitoring unit about the electrical activity changes.

18. The system of claim 17, wherein the reservoir is implantable on or within the subject.

19. The system of claim 17, wherein the monitoring unit comprises a microprocessor that monitors electrical activity and detects eating and/or generates electrical signals based on the monitored electrical activity.

20. The system of claim 17, further comprising a recording module for recording electrical data based on the monitored electrical activity.

21. The system of claim 17, further comprising a pulse generator and a recording module.

22. The system of claim 17, further comprising a computer for receiving electrical signals, analyzing electrical signals, processing electrical signals, and sending a signal regarding the electrical signals to another system, computer or device.

23. The system of claim 17, individualized for each subject.

24. The system of claim 17, further comprising one or more electrodes for detecting the electrical activity changes and positionable within, in contact with or proximate to the gastroesophageal junction of the subject.

25. The system of claim 17, further comprising one or more pumps.

26. The system of claim 17, further comprising a circular wheel and a watch-type drive mechanism that drives the circular wheel, and wherein the wheel contains a plurality of cavities, all of which are radially disposed in a single diametral plane about the circumference of the wheel.
